(19) United States
(12) Patent Application Publication
(10) Pub. No.: US 2008/0281378 A1
(43) Pub. Date: Nov. 13, 2008

(54) Neurological diagnostic and therapeutic system utilizing function-specific modules

(76) Inventors: Michael Williams, Melrose, MA (US); Shai Gozani, Brookline, MA (US); John D'Arco, Wilmington, MA (US)

Correspondence Address: Mark J. Pandiscio;Pandiscio & Pandiscio, P.C., 470 Totten Pond Road, Waltham, MA, 02451-1914, US

(21) Appl. No.: 12/002,039

(22) Filed: Dec. 14, 2007

Related U.S. Application Data
(60) Provisional application No. 60/875,292, filed on Dec. 15, 2006.

(51) Int. Cl. A61N 1/08

(52) U.S. Cl. 607/60

(57) ABSTRACT

A neurological diagnosis and/or treatment system comprising:
(i) patient interface apparatus for interfacing with the patient so as to acquire desired data from the patient;
(ii) a function-specific module for performing a desired diagnostic and/or therapeutic function on the patient via the patient interface apparatus;
(iii) a controlling device for providing a user interface between a medical professional operating the system and the function-specific module, whereby to enable the medical professional to provide input to, and receive output from, the function-specific module; and
(iv) a communications hub for connecting the controlling device with a documentation/analysis/storage center by means of a communications network;
wherein the function-specific module is connected to the patient interface apparatus by a communications link, the function-specific module is connected to the controlling device by a communications link, and the controlling device is connected to the communications hub by a communications link.

A method for treating a patient, comprising:
providing a neurological diagnosis and/or treatment system comprising:
   (i) patient interface apparatus for interfacing with the patient so as to acquire desired data from the patient;
   (ii) a function-specific module for performing a desired diagnostic and/or therapeutic function on the patient via the patient interface apparatus;
   (iii) a controlling device for providing a user interface between a medical professional operating the system and the function-specific module, whereby to enable the medical professional to provide input to, and receive output from, the function-specific module; and
   (iv) a communications hub for connecting the controlling device with a documentation/analysis/storage center by means of a communications network;
   wherein the function-specific module is connected to the patient interface apparatus by a communications link, the function-specific module is connected to the controlling device by a communications link, and the controlling device is connected to the communications hub by a communications link;
applying the patient interface apparatus to the patient;
using the controlling device to provide input to the function-specific module; and
using the controlling device to receive output from the function-specific module.

### REFERENCE TO PENDING PRIOR PATENT APPLICATION

This patent application claims benefit of pending prior U.S. Provisional Patent Application Ser. No. 60/875,292, filed Dec. 15, 2006 by Michael Williams et al. for NEUROLOGICIAL DIAGNOSTIC AND THERAPEUTIC SYSTEM WITH WIRELESS FUNCTIONAL MODULES (Attorney's Docket No. NEURO-22 PROV), which patent application is hereby incorporated herein by reference.

### BACKGROUND OF THE INVENTION

Neurodiagnostic testing, such as nerve conduction studies (NCS) and needle electromyography (nEMG), has traditionally been performed by large, cart-mounted equipment that is operated by specially-trained medical personnel. An example of such equipment is the Viking II System manufactured by Viasys Corporation (Conshohocken, Pa., USA).

These cart-mounted systems are generally multi-functional in nature and provide a diverse range of neurodiagnostic procedures including, but not limited to, nerve conduction studies, needle electromyography, evoked neuromuscular potentials, electroencephalography, intra-operative monitoring, etc. However, by virtue of their substantial size, complexity, cost, and lack of portability, such cart-mounted systems are generally not readily usable at the typical point-of-service, such as in the offices of internists and orthopedic surgeons. However, there is a substantial need for neurodiagnostic systems in these typical point-of-service settings.

As a result, small form-factor, portable devices have been introduced into the marketplace which are better suited for assessing neuromuscular function in physician offices and small clinic settings. These new devices are also designed to be used by personnel who may lack the specialized training generally required by traditional neurodiagnostic equipment. The apparatus and method described in U.S. Pat. No. 5,976,094, issued Nov. 2, 1999 to Gozani for APPARATUS AND METHODS FOR ASSESSMENT OF NEUROMUSCULAR FUNCTION, which patent is hereby incorporated herein by reference, is one example of such a system. This system is commercially available from NeuroMetrix, Inc. (Waltham, Mass., USA) under the tradename NC-stat®.

The NC-stat® system is successfully used many thousands of times every year to assess neuromuscular function. The NC-stat® system generally comprises a biosensor array comprising stimulation and detection electrodes (FIG. 1) which is applied to the patient, a handheld controlling device (FIG. 2) which sends electrical stimuli to the biosensor array and collects electrical responses (i.e., the test data) from the patient, and a communications hub (FIG. 3) which connects the controlling device to a data documentation/analysis/storage center via a telecommunications network.

Although the NC-stat® system is a significant improvement over traditional neurodiagnostic testing equipment, the design of the currently-available NC-stat® system limits its application.

More particularly, the currently-available NC-stat® system uses a controlling device which is dedicated to a specific set of nerve conduction tests (e.g., surface-based, peripherally-located nerve conduction testing). Therefore, a different controlling device must be provided if a different set of neurological tests is to be performed.

Furthermore, the currently-available NC-stat® system must be performed through a cable which connects the controlling device to the biosensor array and, once the data is collected from the patient, the controlling device must be physically connected (via a hard dock) with the communications hub. The communications hub is in turn physically connected (via a wire) with a telecommunications network, in order for the test data to be uploaded from the controlling device to the data documention/analysis/storage center for documentation and/or further analysis and/or storage. Due to the hard-wired nature of the currently-available NC-stat® system, this generally requires that the testing professional leave the patient and physically carry the controlling device to the communications hub for data uploading, thereby taking up valuable professional time and preventing the controlling device from being used to perform another test while the controlling device is away from the patient area.

Wireless biomonitoring is commonly used for remote monitoring of EKG, blood pressure, oxygen saturation, and other common physiological parameters. For example, Welch Allyn (Beaverton, Oreg., USA) offers the FlexNet™ monitoring system with two-way communication to monitor patient vital signs. In another example, Philips offers a Holter monitoring system for monitoring EKG. However, the Holter EKG monitoring is a batch data collection process that provides no remote real-time functionality. In essence, Holter systems passively record EKG waveforms over a time period and the data is later processed. In addition, recuperative and ambulatory EKG is often monitored with wireless telemetry apparatus. This is real-time monitoring, but it is specific to the monitoring of EKG signals.

As noted above, neurodiagnostic testing is generally performed with large, dedicated, hard-wired, cart-mounted equipment. This is because traditional neurodiagnostic testing is typically a one-time test that is completed in one session in the neurologist's office. With the exception of specialized in-patient EKG monitoring, traditional neurodiagnostic testing does not generally require wireless capability, and hence traditional neurodiagnosic equipment does not provide the same.

Thus there is a need to provide a new and improved neurological diagnostic and therapeutic system which addresses the aforementioned functionality constraints and physical connection constraints of the prior art.

### SUMMARY OF THE INVENTION

The present invention addresses both (i) the functionality constraints of the currently-available NC-stat® system, and (ii) the need to physically connect the controlling device with the patient-contacting apparatus (e.g., the biosensor array) in order to obtain the patient test data, and the need to physically connect the controlling device with the communications hub in order to upload the test data.

More particularly, with the present invention, the functionality constraints of the currently-available NC-stat® system are overcome through the use of function-specific modules which are interposed between the patient-contacting apparatus (e.g., the biosensor arrays) and the controlling device so that the system may be used for an increased range of neurological diagnostic and therapeutic tests.

Furthermore, the physical connection limitations of the currently-available NC-stat® system are overcome through the use of a wireless controlling device, a wireless communications hub, and various wireless functional modules. The present invention can also use wireless apparatus (e.g., wireless biosensor arrays) for interfacing with the patient. Among other things, radio frequency (RF) links and/or optical links (e.g., infrared light) may be used to provide the wireless communications.

The novel wireless neurological diagnostic and therapeutic system of the present invention is capable of performing a diverse range of different function-specific tests, and is capable of providing the portability needed for the dynamic nature of the point-of-service environment in which the system is to be used.

Specifically, the system provides a variety of different, function-specific diagnostic/therapeutic modules for providing increased functionality, and a controlling device having a universal user interface for operating the function-specific modules, regardless of which one (or ones) of the particular function-specific module are being used.

Additionally, the present invention utilizes wireless connections to interconnect the individual system components, whereby to wirelessly transfer the test data. More particularly, the present invention wirelessly transfers test data from the patient to the function-specific module, and/or to the controlling device, and/or to the communications hub, from which it is sent to the documentation/analysis/storage center.

In addition, unlike the more traditional physical parameters commonly monitored in a hospital ICU, neurological tests generally require real-time, dynamic control during the duration of the test. For example, nerve conduction studies (NCS) typically require that the stimulus current and pulse width be controlled for the duration of the test. These real-time, dynamic control requirements necessitate a significantly more complex wireless communications link when applied to such neurological testing. This is in stark contrast to the relatively simple physical parameters more traditionally monitored in a hospital, e.g., patient vital signs, etc.

The present invention also provides a flexible system that eliminates the need to supply individual controlling devices for each separate function which is to be provided by the system. Furthermore, the present invention also eliminates the need to repetitively enter patient data for each different function provided by the system, thereby keeping medical costs lower.

In one preferred embodiment, the present invention utilizes a wireless controlling device that provides the user interface and computational test date processing capabilities. The controlling device interacts in a real-time fashion with a variety of wireless, function-specific modules, each of which performs specialized neurological functions, including those of a diagnostic and therapeutic nature. The controlling device also communicates, through a wireless link, with a fixed communications hub which may be physically attached to a telecommunications infrastructure such as a landline or cellular telephone network or the Internet.

In one preferred form of the invention, there is provided a neurological diagnosis and/or treatment system comprising:

(i) patient interface apparatus for interfacing with the patient so as to acquire desired data from the patient;

(ii) a function-specific module for performing a desired diagnostic and/or therapeutic function on the patient via the patient interface apparatus;

(iii) a controlling device for providing a user interface between a medical professional operating the system and the function-specific module, whereby to enable the medical professional to provide input to, and receive output from, the function-specific module; and

(iv) a communications hub for connecting the controlling device with a documentation/analysis/storage center by means of a communications network;

wherein the function-specific module is connected to the patient interface apparatus by a communications link, the function-specific module is connected to the controlling device by a communications link, and the controlling device is connected to the communications hub by a communications link.

In another preferred form of the present invention, there is provided a method for treating a patient, comprising:

providing a neurological diagnosis and/or treatment system comprising:
(i) patient interface apparatus for interfacing with the patient so as to acquire desired data from the patient;
(ii) a function-specific module for performing a desired diagnostic and/or therapeutic function on the patient via the patient interface apparatus;
(iii) a controlling device for providing a user interface between a medical professional operating the system and the function-specific module, whereby to enable the medical professional to provide input to, and receive output from, the function-specific module; and
(iv) a communications hub for connecting the controlling device with a documentation/analysis/storage center by means of a communications network;
wherein the function-specific module is connected to the patient interface apparatus by a communications link, the function-specific module is connected to the controlling device by a communications link, and the controlling device is connected to the communications hub by a communications link;

applying the patient interface apparatus to the patient;

using the controlling device to provide input to the function-specific module; and

using the controlling device to receive output from the function-specific module.

### BRIEF DESCRIPTION OF THE DRAWINGS

FIG. 1 is a schematic view showing a biosensor array from a currently-available NC-stat® system;

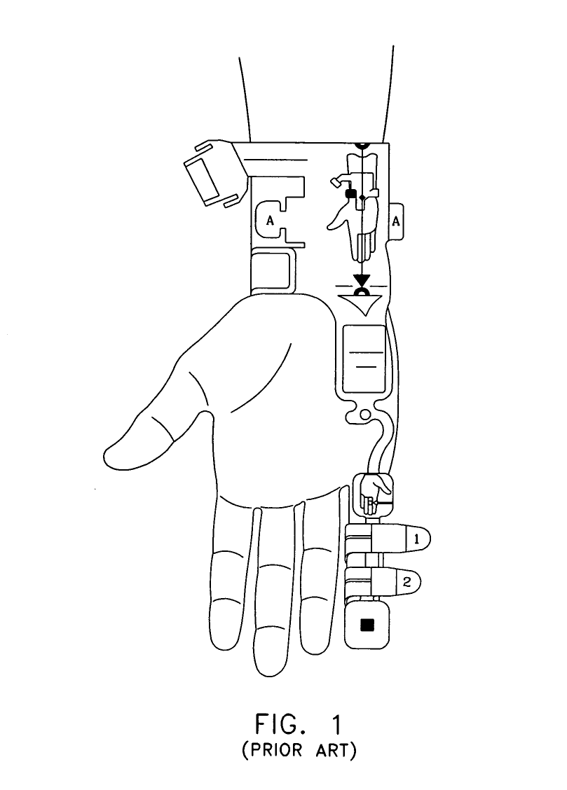

FIG. 2 is a schematic view showing a controlling device from a currently-available NC-stat® system;

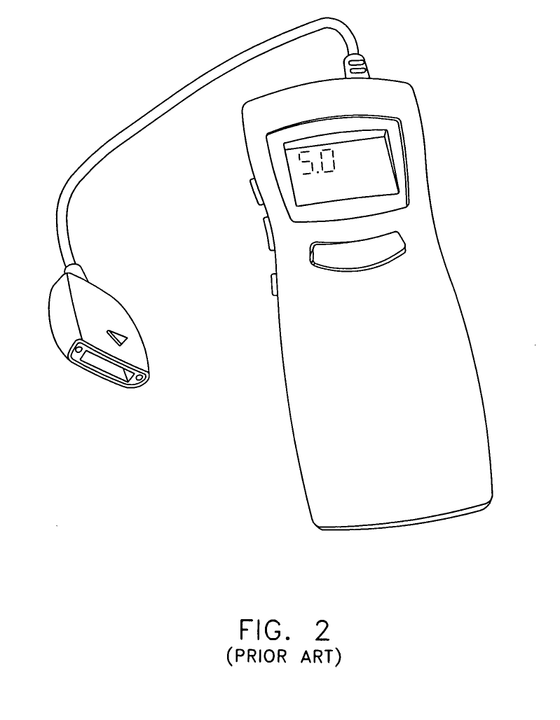

FIG. 3 is a schematic view showing a communications device from a currently-available NC-stat® system;

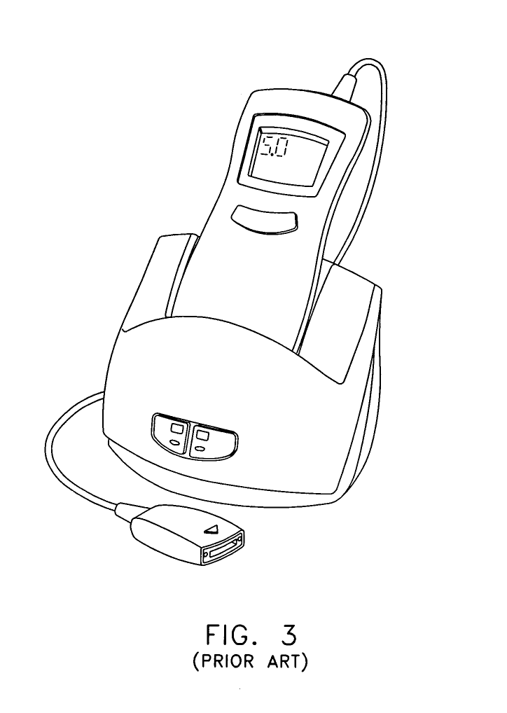

FIG. 4 is a schematic view showing a novel neurological diagnosis and treatment system formed in accordance with the present invention;

FIG. 5 is a schematic view showing a wireless controlling device wirelessly controlling a wireless functional module, wherein the wireless functional module provides a specialized neurological testing functionality; and

FIG. 6 is a schematic view showing the wireless controlling device of FIGS. 4 and 5 wirelessly transferring patient data to a wireless communications hub, which is in turn connected (either wirelessly or with hard wire) to a communications network, e.g., a landline or cellular telephone system or the Internet.

### DETAILED DESCRIPTION OF THE INVENTION

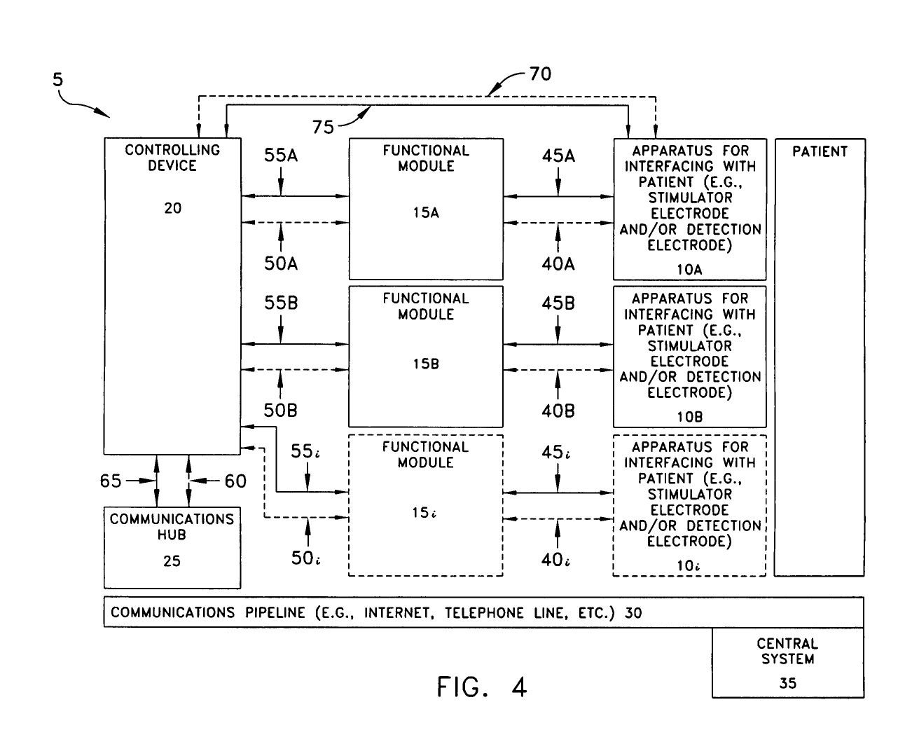

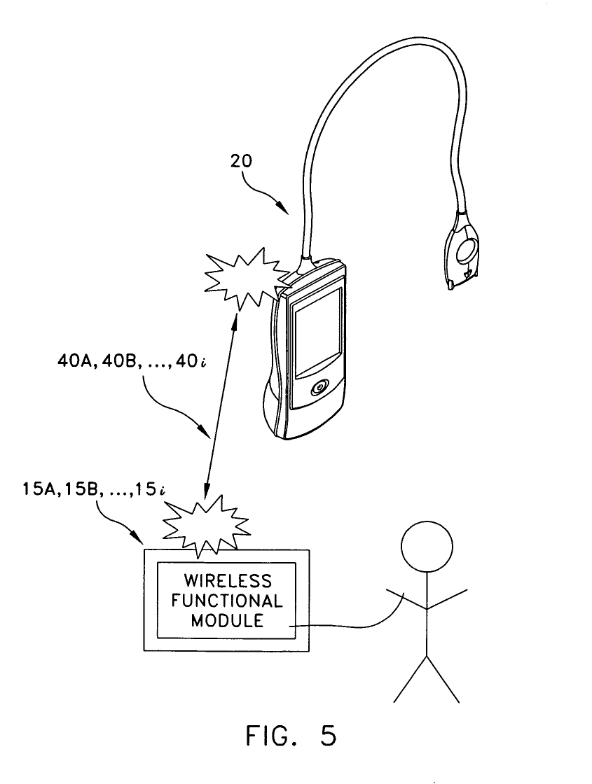

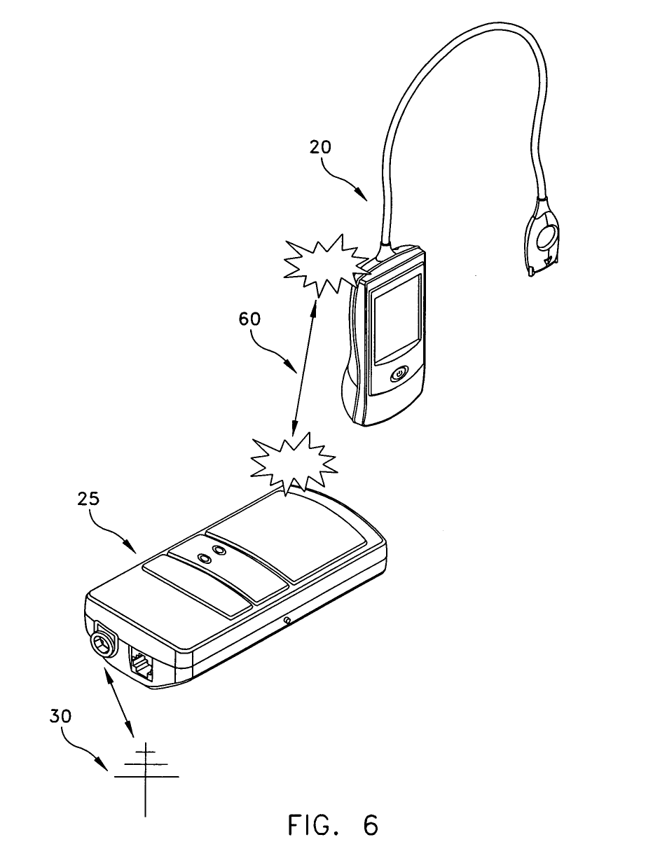

Looking now at FIGS. 4-6, there is shown a novel neurological diagnosis and treatment system 5 formed in accordance with the present invention. Neurological diagnosis and treatment system 5 is capable of performing a variety of different diagnostic and therapeutic procedures including, but not limited to, nerve conduction studies (NCS), needle electromyography (nEMG), other peripheral nerve diagnostic tests, peripheral nerve therapeutic procedures, etc., and utilizes wireless links to connect together one or more of its components.

More particularly, the novel neurological diagnosis and treatment system 5 comprises (i) patient interface apparatus 10A, 10B, . . . , 10i for interfacing with the patient so as to acquire desired data from the patient (e.g., a biosensor array comprising stimulation and detection electrodes, etc.), (ii) one or more function-specific modules (“functional modules”) 15A, 15B, . . . , 15i for performing the desired diagnostic or therapeutic functions via the patient interface apparatus, (iii) a controlling device 20 for providing the user interface between the medical professional and the functional modules, whereby to provide control commands and information (e.g., configuration information, functional parameters, etc.) to the functional modules, and (iv) a communications hub 25 for connecting the controlling device with the documentation/analysis/storage center 30 by means of a communications network 35. Function-specific modules 15A, 15B, . . . , 15i, and in some cases patient interface apparatus 10A, 10B, . . . , 10i, will vary according to the specific procedure which is to be performed by the system. By way of example but not limitation, where system 5 is to provide a nerve conduction study (NCS), patient interface apparatus 10A may comprise a biosensor array comprising stimulation and detection electrodes, and functional module 15A may comprise the hardware and software necessary to provide nerve conduction studies. By way of further example but not limitation, where system 5 is to provide needle electromyography (nEMG), patient interface apparatus 10B may comprise the necessary needle electrodes, and functional module 15B may comprise the hardware and software necessary to provide the electromyography studies.

The various components of system 5 are preferably connected together by wireless communication links, although hardwired connections may also be used. More particularly, function module 15A, 15B, . . . , 15i are preferably connected to patient interface apparatus 10A, 10B, . . . , 10 i by wireless links 40A, 40B, . . . , 40i, respectively, although they could also be connected by hardwired links 45A, 45B, . . . , 45i, respectively. Furthermore, function module 15A, 15B, . . . , 15i are preferably connected to controlling device 20 by wireless links 50A, 50B, . . . , 50i, respectively, although they could also be connected by hardwired links 55A, 55B, . . . , 55i, respectively. Additionally, controlling device 20 is preferably connected to communications hub 25 by wireless link 60, although it could also be connected by hardwired link 65.

In one preferred form of the present invention, diagnostic tests may be performed in a mode whereby controlling device 20 is used essentially only for its user interface and computational processing capabilities; in this mode, the primary diagnostic functions are provided by the one or more wireless modules 15A, 15B, . . . , 15i. By way of example but not limitation, controlling device 20 may be used to wirelessly control one or more specialized wireless modules 15A, 15B, . . . , 15i which may in turn be connected (wirelessly or by hardwire) to patient interface apparatus 10A, 10B, . . . , 10i (e.g., a biosensor array, etc.), and controlling device 20 may be connected (wirelessly or by hard wire) to other system components (e.g., to communications hub 25).

In another configuration, diagnostic tests may be performed in a mode whereby the embedded diagnostic capabilities of controlling device 20 are used in conjunction with one more wireless modules 15A, 15B, . . . , 15i.

In another configuration, controlling device 20 is used with one or more wireless modules 15A, 15B, . . . , 15i providing therapeutic functionality. In this mode, the embedded functions of the controlling device may or may not be used in conjunction with the wireless therapeutic modules 15A, 15B, . . . , 15i.

In yet another embodiment of the present invention, diagnostic and therapeutic interventions involving the central nervous system are provided.

The aforementioned wireless functional modules 15A, 15B, . . . , 15i may be configured so as to provide a variety of different functions. By way of example but not limitation, the novel system 5 may incorporate one or more of the following wireless functional modules 15A, 15B, . . . , 15i.

1. Proximal Nerve Stimulation Module (Proximal). This functional module consists of a battery-operated device with a high voltage stimulator and at least one stimulation channel. This module interfaces with the patient through a patient interface apparatus which comprises at least two stimulation electrodes. The functional module receives stimulation parameters and triggers from the controlling device via a wireless link. The functional module sends stimulation status data back to the controlling device via the wireless link. Thus, this wireless functional module is effectively a wireless biosensor array.

2. Needle Electromyography Module (nEMG). This functional module consists of a battery-operated device that interfaces with a patient through a patient interface apparatus which comprises a disposable needle electrode and a surface electrode. This functional module records, amplifies, and digitizes electromyographic signals from skeletal muscle and transmits these signals via a wireless link to the controlling device. This transmission is performed in real-time so that a physician can, for example, visualize the signals on the controlling device's LCD display. This functional module also accepts configuration parameters such as speaker volume, amplifier gain, and filter settings from the controlling device through the wireless link.

3. Cardiac Autonomic Neuropathy Module (CANS). This functional module consists of a battery-operated device that records, amplifies, processes, and digitizes three-lead (or other number of leads that will reliably produce beat-to-beat timing information) EKG signals obtained from a patient and transmits both raw and processed signals via a wireless link to the controlling device. The CANS module interfaces with the patient through a patient interface apparatus which comprises at least three surface electrodes. This functional module receives configuration parameters from the controlling device via the wireless link.

4. Near Nerve Injection Module (NNI). This functional module is a device that detects the proximity of a needle to a target nerve through measurement and analysis of nerve-evoked responses. The NNI module transmits its ongoing status and final nerve proximity data through the wireless link to the controlling device. This functional module receives configuration parameters from the controlling device through the wireless link. This test is performed for the purpose of accurately locating a needle, in real-time, in very close proximity to a target nerve, for the purpose of delivering a therapeutic agent to the target nerve, and/or for diagnostic purposes. Thus, in this form of the invention, the patient interface apparatus comprises at least one needle.

It should be appreciated that each system 5 may comprise a single function-specific module 15A, 15B, . . . , 15i, or it may comprise a plurality of function-specific modules 15A, 15B, . . . , 15i depending on which diagnostic or therapeutic procedures are to be conducted on the patient.

Furthermore, the type and number of patient interface devices 10A, 10B, . . . , 10i provided in system 5 is consistent with the number and type of function-specific modules 15A, 15B, . . . , 15i provided in the system.

It should also be appreciated that, in some circumstances, a single patient interface apparatus 10A, 10B, . . . , 10i may be used by a plurality of function-specific modules 15A, 15B, . . . , 15i; and, in some circumstances, a single function-specific module 15A, 15B, . . . , 15i may use a plurality of patient interface apparatus 10A, 10B, . . . , 10i.

The wireless controlling device 20 and wireless functional modules 15A, 15B, . . . , 15i engage in a two-way communication whereby the controlling device 20 wirelessly sends control commands and information (e.g., configuration information, functional parameters, etc.) to the modules 15A, 15B, . . . , 15i, and the modules 15A, 15B, . . . , 15i wirelessly send status, and raw and processed data, back to the controlling device 20.

Real-time analysis may be performed by controlling device 20 (or wireless modules 15A, 15B, . . . , 15i) on the collected data, and numerical and graphical results may be provided (via controlling device 20) to on-site medical testing professionals.

Collected data may also be transmitted from controlling device 20 to a wireless communications hub 25, which in turn can upload the data, via a communications system 30 such as a landline or cellular telephone network or the Internet, to a data documention/analysis/storage center 35.

Thus, in one aspect of the invention, there is provided a novel system for neurological testing and/or therapy which uses a wireless master controlling device 20.

And in another aspect of the invention, there is provided a novel system for neurological testing and/or therapy which uses one or more wireless functional modules 15A, 15B, . . . , 15i.

And in still another aspect of the system, there is provided the aforementioned NCS, nEMG, CANS and NNI functionality as wireless functional modules 15A, 15B, . . . , 15i.

It should also be appreciated that controlling device 20 may be configured to be used in a “stand-alone” mode, without any wireless modules. In this stand-alone mode, the diagnostic procedure is performed entirely with the functional capabilities embedded within the controlling device. By way of example but not limitation, controlling device 20 may be connected (via a wireless link 70 or by a hardwire link 75) directly to the patient interface apparatus 10A (e.g., a biosensor array), and controlling device 20 may be wirelessly connected to other system components (e.g., communications hub 25 for data off-loading).

### Modifications

It will be appreciated that still further embodiments of the present invention will be apparent to those skilled in the art in view of the present disclosure. It is to be understood that the present invention is by no means limited to the particular constructions herein disclosed and/or shown in the drawings, but also comprises any modifications or equivalents within the scope of the invention.

What is claimed is:

1. A neurological diagnosis and/or treatment system comprising:
(i) patient interface apparatus for interfacing with the patient so as to acquire desired data from the patient;
(ii) a function-specific module for performing a desired diagnostic and/or therapeutic function on the patient via the patient interface apparatus;
(iii) a controlling device for providing a user interface between a medical professional operating the system and the function-specific module, whereby to enable the medical professional to provide input to, and receive output from, the function-specific module; and
(iv) a communications hub for connecting the controlling device with a documentation/analysis/storage center by means of a communications network;
wherein the function-specific module is connected to the patient interface apparatus by a communications link, the function-specific module is connected to the controlling device by a communications link, and the controlling device is connected to the communications hub by a communications link.

2. A system according to claim 1 wherein the system comprises a plurality of function-specific modules, and further wherein at least two of the function-specific modules utilize the same patient interface apparatus.

3. A system according to claim 1 wherein the system comprises a plurality of plurality of patient interface apparatus and a plurality of function-specific modules.

4. A system according to claim 3 wherein each of the function-specific modules utilizes a separate patient interface apparatus.

5. A system according to claim 1 wherein the function-specific module is adapted to conduct nerve conduction studies by applying an electrical stimulus to a patient and detecting and analyzing a patient response to the stimulus, and further wherein the patient interface apparatus comprises a biosensor having at least one stimulation electrode and at least one detection electrode.

6. A system according to claim 1 wherein the function-specific module is adapted to conduct needle electromyography studies by applying an electrical stimulus to a patient and detecting and analyzing a patient response to the stimulus, and further wherein the patient interface apparatus comprises a needle electrode and a surface electrode.

7. A system according to claim 1 wherein the function-specific module is adapted to conduct cardiac autonomic neuropathy studies by detecting and analyzing a beat-to-beat timing information, and further wherein the patient interface apparatus comprises a plurality of surface electrodes.

8. A system according to claim I wherein the function-specific module is adapted to conduct near nerve conduction studies by applying an electrical stimulus to a patient and detecting and analyzing a patient response to the stimulus, and further wherein the patient interface apparatus comprises at least one needle.

9. A system according to claim 1 wherein real-time analysis is performed on the desired data by the function-specific module.

10. A system according to claim 1 wherein real-time analysis is performed on the desired data by the controlling device.

11. A system according to claim 1 wherein the function-specific module is connected to the patient interface apparatus by a wireless communication link.

12. A system according to claim 1 wherein the function-specific module is connected to the controlling device by a wireless communication link.

13. A system according to claim 1 wherein the controlling device is connected to the communications hub by a wireless communication link.

14. A system according to claim 1 wherein the controlling device is configured to send control commands and information to the function-specific module.

15. A system according to claim 14 wherein the controlling device is configured to send configuration information and functional parameters to the function-specific module.

16. A method for treating a patient, comprising:
providing a neurological diagnosis and/or treatment system comprising:
(i) patient interface apparatus for interfacing with the patient so as to acquire desired data from the patient;
(ii) a function-specific module for performing a desired diagnostic and/or therapeutic function on the patient via the patient interface apparatus;
(iii) a controlling device for providing a user interface between a medical professional operating the system and the function-specific module, whereby to enable the medical professional to provide input to, and receive output from, the function-specific module; and
(iv) a communications hub for connecting the controlling device with a documentation/analysis/storage center by means of a communications network;
wherein the function-specific module is connected to the patient interface apparatus by a communications link, the function-specific module is connected to the controlling device by a communications link, and the controlling device is connected to the communications hub by a communications link;
applying the patient interface apparatus to the patient;
using the controlling device to provide input to the function-specific module; and
using the controlling device to receive output from the function-specific module.

17. A method according to claim 16 wherein the function-specific module is adapted to conduct nerve conduction studies by applying an electrical stimulus to a patient and detecting and analyzing a patient response to the stimulus, and further wherein the patient interface apparatus comprises a biosensor having at least one stimulation electrode and at least one detection electrode.

18. A method according to claim 16 wherein the function-specific module is adapted to conduct needle electromyography studies by applying an electrical stimulus to a patient and detecting and analyzing a patient response to the stimulus, and further wherein the patient interface apparatus comprises a needle electrode and a surface electrode.

19. A method according to claim 16 wherein the function-specific module is adapted to conduct cardiac autonomic neuropathy studies by detecting and analyzing a beat-to-beat timing information, and further wherein the patient interface apparatus comprises a plurality of surface electrodes.

20. A method according to claim 16 wherein the function-specific module is adapted to conduct near nerve conduction studies by applying an electrical stimulus to a patient and detecting and analyzing a patient response to the stimulus, and further wherein the patient interface apparatus comprises at least one needle.

21. A method according to claim 16 wherein real-time analysis is performed on the desired data by the function-specific module.

22. A method according to claim 16 wherein real-time analysis is performed on the desired data by the controlling device.

23. A method according to claim 16 wherein the function-specific module is connected to the patient interface apparatus by a wireless communication link.

24. A method according to claim 16 wherein the function-specific module is connected to the controlling device by a wireless communication link.

25. A method according to claim 16 wherein the controlling device is connected to the communications hub by a wireless communication link.
